SEQUENCE LISTING (4 sequences)

SEQ ID NO: 1 - DNA, 10 bp, synthetic construct
  source 1..10: mol_type = other DNA
  misc_feature 1..10: note = synthetic ss DNA linker
gactagtctt

SEQ ID NO: 2 - DNA, 17 bp, synthetic construct
  source 1..17: mol_type = other DNA
  misc_feature 1..17: note = synthetic ss DNA linker
  misc_feature 17: note = where N is thymidine bearing triethyleneglycol-linked cholesterol
tacctacactccaattn

SEQ ID NO: 3 - DNA, 17 bp, synthetic construct
  source 1..17: mol_type = other DNA
  misc_feature 1..17: note = synthetic ss DNA linker
attggagtgtaggtatt

SEQ ID NO: 4 - DNA, 35 bp, synthetic construct
  source 1..35: mol_type = other DNA
  misc_feature 1..35: note = synthetic ss DNA linker
attggagtgtaggtatttttttttttttttttttt

(19) United States
(12) Patent Application Publication
(10) Pub. No.: US 2026/0265734 A1
(43) Pub. Date: Sep. 10, 2026

(54) FLEXIBLE ANTIVIRAL DNA ORIGAMI SHELLS THAT BLOCK INTERACTIONS BETWEEN VIRUS AND HOST CELLS

(71) Applicant: BRANDEIS UNIVERSITY, Waltham, MA (US)

(72) Inventors: Seth FRADEN, Newton, MA (US); W. Benjamin ROGERS, Waltham, MA (US); Tijana IVANOVIC, Waltham, MA (US)

(21) Appl. No.: 18/873,614

(22) PCT Filed: Jun. 14, 2023
(86) PCT No.: PCT/US23/68418

§ 371 Date: Dec. 10, 2024

Related U.S. Application Data
(60) Provisional application No. 63/352,200, filed on Jun. 14, 2022.

(51) Int. Cl. C12N 15/11; A61P 31/14

(52) U.S. Cl. CPC C12N 15/11; A61P 31/14; C12N 2310/3513; C12N 2310/532; C12N 2320/32

(57) ABSTRACT

The present disclosure relates to three-dimensional nucleic acid origami nanostructures that are designed to allow for flexible self-assembly of the nanostructures into a larger structure (e.g., cylindrical, icosahedral, etc.) about the surface of a virus to encapsulate the virus, compositions comprising the nanostructures, and their use in treatment methods.

This application claims the priority benefit of U.S. Provisional Patent Application Ser. No. 63/352,200, filed Jun. 14, 2022, which is hereby incorporated by reference in its entirety.

The Sequence Listing is being submitted electronically in XML format and is hereby incorporated by reference in its entirety. Said XML copy, created on Jun. 13, 2023, is named 147376.000721.xml and is 6.39 KB in size. No new matter is being introduced.

### FIELD OF THE INVENTION

The present disclosure relates to DNA origami subunits that flexibly attach to one another to form shells, and their use for encapsulation of viruses.

### BACKGROUND OF THE INVENTION

For the majority of viral diseases, no effective treatment is available. Broadly applicable antiviral platform technologies do not exist.

Protein designers have previously succeeded in creating artificial macromolecular cages (Bale et al., “Accurate Design of Megadalton-Scale Two-Component Icosahedral Protein Complexes,” Science 353:389-394 (2016); King et al., “Accurate Design of Co-Assembling Multi-Component Protein Nanomaterials,” Nature 510:103-108 (2014); Lai et al., “Structure of a Designed Protein Cage that Self-Assembles into a Highly Porous Cube,” Nature Chemistry 6:1065-1071 (2014); Butterfield et al., “Evolution of a Designed Protein Assembly Encapsulating its Own RNA Genome,” Nature 552:415-420 (2017)). However, the designed protein-cages are much smaller than the vast majority of natural viruses and cannot be easily modified. DNA nanotechnology (Rothemund, “Folding DNA to Create Nanoscale Shapes and Patterns,” Nature 440:297-302 (2006); Douglas et al., “Self-Assembly of DNA into Nanoscale Three-Dimensional Shapes,” Nature 459:414-418 (2009); Castro et al., “A Primer to Scaffolded DNA Origami,” Nature Methods 8:221-229 (2011); Veneziano et al., “Designer Nanoscale DNA Assemblies Programmed from the Top Down,” Science 352:1534 (2016); Benson et al., “DNA Rendering of Polyhedral Meshes at the Nanoscale,” Nature 523:441-444 (2015); Dunn et al., “Guiding the Folding Pathway of DNA Origami,” Nature 525:82-86 (2015)) can create discrete objects with structurally well-defined 3D shapes (Bai et al., “Cryo-EM Structure of a 3D DNA-Origami Object,” PNAS 109:20012-20017 (2012); Funke et al., “Placing Molecules with Bohr Radius Resolution Using DNA Origami,” Nature Nanotechnology 11:47-52 (2016)), including higher-order objects (Iinuma et al., “Polyhedra Self-Assembled from DNA Tripods and Characterized with 3D DNA-PAINT,” Science 344:65-69 (2014); Jungmann et al., “DNA Origami-Based Nanoribbons: Assembly, Length Distribution, and Twist,” Nanotechnology 22:275301 (2011); Liu et al., “Crystalline Two-Dimensional DNA-Origami Arrays,” Angewandte Chemie 50:264-267 (2011); Suzuki et al., “Lipid-Bilayer-Assisted Two-Dimensional Self-Assembly of DNA Origami Nanostructures,” Nature Communications 6:8052 (2015); Ke et al., “DNA Brick Crystals with Prescribed Depths,” Nature Chemistry 6:994-1002 (2014)) with molecular masses exceeding one Gigadalton (Wagenbauer et al., “Gigadalton-Scale Shape-Programmable DNA Assemblies,” Nature 552:78-83 (2017)). However, these previous designs and the underlying concepts yield objects that are either too small, assemble with insufficient yields, do not match the shapes of viruses, or are too inflexible or skeletal to be suitable for effectively trapping and occluding entire virus particles.

The present disclosure is directed to overcoming these and other deficiencies in the art.

### SUMMARY OF THE INVENTION

One aspect of the present disclosure relates to a three-dimensional DNA molecular structure comprising one or more DNA strands folded in the form of a nanoscale triangular subunit having a configuration that allows a plurality of said nanoscale triangular subunits to self-assemble in the form of a flexible macromolecular shell, wherein the nanoscale triangular subunits are connected together by hybridization of one or more single-stranded DNA (“ss-DNA”) linkers on adjoining triangular subunits, which retain a number of unpaired bases whereby the unpaired bases of the hybridized ss-DNA linkers enables the macromolecular shell to bend and stretch around a virus or other particle intended for encapsulation.

Another aspect of the present disclosure relates to flexible macromolecular shell formed by self-assembly of a plurality of the three-dimensional DNA molecular structures described herein.

A further aspect of the present disclosure relates to a composition comprising a plurality of three-dimensional DNA molecular structures described herein in a carrier.

Another aspect of the present disclosure relates to a composition comprising a plurality of flexible macromolecular shells described herein in a carrier.

A further aspect of the present disclosure relates to a composition comprising a plurality of three-dimensional DNA molecular structures described herein and a plurality of flexible macromolecular shells as described herein in a carrier.

Another aspect of the present disclosure relates to a method of encapsulating a virus. This method involves providing a plurality of the three-dimensional DNA molecular structures described herein, and allowing said three-dimensional DNA molecular structures to self-assemble around a virus to form a shell, thereby encapsulating the virus.

A further aspect of the present disclosure relates to a method of inhibiting viral infection. This method involves encapsulating a virus with a flexible macromolecular shell as described herein, whereby the shell forms a physical barrier to inhibit viral infection of a cell otherwise susceptible to infection by the virus.

Another aspect of the present disclosure relates to a method of treating an individual for a viral infection. This method involves administering a composition described herein to an individual at a site of viral infection, where the flexible macromolecular shell forms a physical barrier that encapsulates viruses at the site of viral infection, thereby treating the individual.

The present disclosure relates to trapping entire virus particles within de novo designed macromolecular shells to inhibit molecular interactions between viruses and host cells. Flexible shells are used to augment and work synergistically with a large variety of virus binding moieties, whether by themselves neutralizing or not, to create an effective antiviral agent.

To accomplish this function, flexible shells are made that are large enough and bendable or flexible or stretch enough to accommodate entire viruses and their unique or non-uniform shapes, while also being chemically addressable to allow including virus-specificity conferring moieties on the shell's interior surface. The extended surface of the shells enables functionalization in a multivalent fashion. Multivalency can support tight binding of a target virus even for individually weakly virus-binding molecules, as exemplified in previous experiments with phage nanoparticles engineered to trivalently bind influenza A hemagglutinin (Lauster et al., “Phage Capsid Nanoparticles with Defined Ligand Arrangement Block Influenza Virus Entry,” Nature Nanotechnology 15:373-379 (2020), which is hereby incorporated by reference in its entirety), and with star-shaped DNA aptamer clusters that simultaneously target multiple dengue virus envelope proteins (Kwon et al., “Designer DNA Architecture Offers Precise and Multivalent Spatial Pattern-Recognition for Viral Sensing and Inhibition,” Nature Chemistry 12:26-35 (2020), which is hereby incorporated by reference in its entirety). With shells that fully cover viruses, an even larger degree of multivalency, and thus stronger binding, is envisioned. Modular functionalization of the shells with virus binders will enable using the same type of shell platform to target a variety of viruses. Candidate virus binders could be, e.g., antibodies, designed proteins (Cao et al., “De Novo Design of Picomolar SARS-CoV-2 Miniprotein Inhibitors,” Science 370:426-431 (2020), which is hereby incorporated by reference in its entirety), nucleic acid aptamers, or polymers such as heparan sulphate (Cagno et al., “Sulfate Proteoglycans and Viral Attachment: True Receptors or Adaptation Bias?” Viruses 11 (2019), which is hereby incorporated by reference in its entirety). The shell material, rather than the moieties directly contacting the virus, will mainly prevent access to the viral surface. Therefore, in principle any virus binding molecule could potentially be utilized to convert the shells into an effective virus-neutralizing trap.

The shell concept described herein requires constructing massive molecular complexes that are adaptable to cover the dimensions of viral pathogens (~20 nm to ~500 nm) (see Legendre et al., “Thirty-Thousand-Year-Old Distant Relative of Giant Icosahedral DNA Viruses with a Pandoravirus Morphology,” PNAS 111:4274-4279 (2014), which is hereby incorporated by reference in its entirety), which poses a fundamental nanoengineering challenge.

While past DNA origami structures have been formed using rigid triangular subunits, the use of one or more ss-DNA linkers allows for flexibly linking together of a plurality of triangular subunits. The ss-DNA linkers are used in conjunction with a staple strand that creates an anchor that attaches one end of the ss-DNA linker to the triangular subunits. Each ss-DNA linker includes a hybridizing segment at the other end of the ss-DNA linker, and a spacer segment between the anchor and the hybridizing segment. The spacer segment length controls the spacing, flexibility, and degree of bending between a pair of triangular subunits that are joined together due to hybridization between mating hybridizing segments. As demonstrated herein, the length of the hybridizing segments can be optimized to promote assembly and the length of the spacer segments can be optimized to facilitate flexibility to accommodate irregularly shaped viruses.

To build the envisioned virus trap, a programmable flexible, bendable, stretchable shell “canvas” was created by adapting symmetry principles known from natural viral capsids. Caspar and Klug elucidated the geometric principles that govern the structure of natural viral capsids in 1962 (Caspar et al., “Physical Principles in the Construction of Regular Viruses,” Cold Spring Harbor Symposia on Quantitative Biology 27:1-24 (1962), which is hereby incorporated by reference in its entirety). According to Caspar and Klug theory, which has been expanded recently (Twarock et al., “Structural Puzzles in Virology Solved with an Overarching Icosahedral Design Principle,” Nature Communications 10:4414 (2019), which is hereby incorporated by reference in its entirety), the number of distinct environments occupied by proteins within an icosahedral capsid is described by its triangulation number (T-number), which can be computed by the arrangement of pentamers and hexamers within an icosahedral capsid (T=h²+hk+k²). The total number of proteins required to build a natural capsid is T times sixty. This is because natural protein subunits are, by default, asymmetric and homo-trimerization is minimally required to construct a three-fold symmetric subunit that can assemble into an icosahedral shell with twenty triangular faces. To build larger capsids, viruses use more than one capsid protein or capsid proteins that can adopt different conformations. The structure of natural virus capsids forms the basis for the synthetic programmable icosahedral shell canvasses described herein, which are analogously classified using a T-number.

### BRIEF DESCRIPTION OF THE DRAWINGS

FIG. 1 is a schematic illustration of a triangular subunit where each side includes a plurality of ss-DNA linkers (five are shown, designated as R, Q, P, Q′, and R′). Hybridization of R and R′, hybridization of Q and Q′, and hybridization of two P linkers will occur.

FIG. 2 is a schematic illustration of the triangular subunit of FIG. 1, and illustrates an exemplary ss-DNA linker having the sequence GACTAGTCTT (SEQ ID NO: 1), which includes an 8-nucleotide sequence for hybridization and a 2-thymidine sequence that remains unpaired.

FIG. 3 illustrates a schematic for self-assembly of triangular subunits and temperature and salt conditions to facilitate optimized self-assembly of triangular subunits. Electrophoresis results confirm structural formation of the triangular subunits.

FIG. 4 graphically illustrates conditions for optimization of self-assembly of triangular subunits for the selected conditions (box drawn around 56-53° C. at 15 mM Mg²⁺, and box drawn around 15 mM Mg²⁺ at 60-44° C.) shown in the electrophoresis results of FIG. 3.

FIG. 5 is an image of a gel illustrating electrophoresis separation of monomer and dimer triangular subunits for subunits having different length hybridizing segments on ss-DNA linkers. Hybridizing segments of 6 and 8 bp both contributed to dimer formation, whereas hybridizing segments of 2 or 4 bp show only background levels of dimer formation. Relative amounts of dimer and monomers for 0 bp versus 8 bp samples are shown in the graph.

FIG. 6 illustrates a proof of principle for dimer and multimeric unit self-assembly on cholesterol particles. Cholesterol particles bearing TACCTACACTCCAATTT-triethyleneglycol (“TEG”)-linked DNA strands (SEQ ID NO:2) were used along with triangular subunits bearing ATTGGAGTGTAGGTATT ss-DNA linker strands (SEQ ID NO:3), which were secured to the triangular subunits using staple strands. The structure to the right shows relative spacing of the ss-DNA linker strands around the perimeter of the triangular subunits. These ss-DNA linker strands facilitate subunit-subunit binding.

FIG. 7 schematically illustrates the assembly of dimer and multimeric units on the surface of cholesterol particles. Depending on the length of the spacer segment in the ss-DNA linker strands, less or greater flexibility can be achieved. Whereas ATTGGAGTGTAGGTATT ss-DNA linker strands (SEQ ID NO: 3) showed very little flexibility due to only a 2-thymidine spacer sequence, ATTGGAGTGTAGGTATTTTTTTTTTTTTTTTTTTT ss-DNA linker strands (SEQ ID NO: 4) showed much greater flexibility due to a 20-thymidine spacer sequence.

FIG. 8 is a transmission electron microscopy (“TEM”) image illustrating dimer assembly on cholesterol particles. Inset is an enlarged image showing a single dimer assembly.

FIGS. 9A-E illustrate a proposed mechanism for partial encapsulation of virus particle using an open origami shell whose triangular subunits are coated with antibodies to a virus. Once trapped inside the shell, the virus is unable to fuse with a cell (FIG. 9A). Negative stain EM images of VSV-CoV2S virus (FIG. 9B), which serves as a mimic for covid-19 to test deactivation. Cryo-EM structure of open DNA origami shell (FIG. 9C), of shell with HBV virus entrapped (FIG. 9D), and 3 HBV virus entrapped (FIG. 9E).

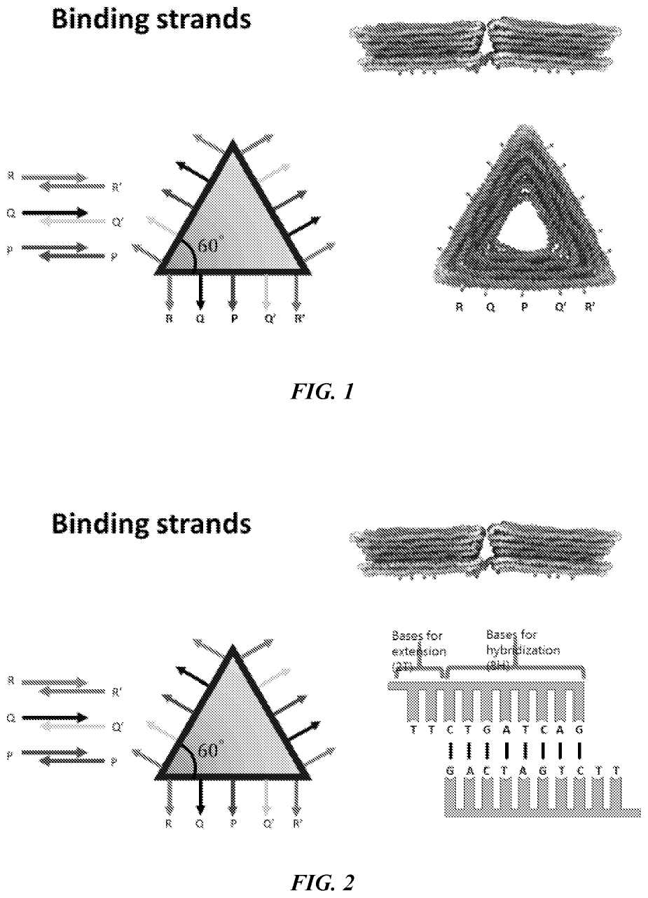

### DETAILED DESCRIPTION OF THE INVENTION

One aspect of the present disclosure relates to a three-dimensional DNA molecular structure comprising one or more DNA strands folded in the form of a nanoscale triangular subunit having a configuration that allows a plurality of said nanoscale triangular subunits to self-assemble in the form of a flexible macromolecular shell, wherein the nanoscale triangular subunits are connected together by hybridization of one or more single-stranded DNA (ss-DNA) linkers on adjoining triangular subunits, which retain a number of unpaired bases whereby the unpaired bases of the hybridized ss-DNA linkers enables the macromolecular shell to bend and stretch around a virus or other particle intended for encapsulation.

As referred to herein, DNA (or, more broadly, nucleic acid molecules, including deoxyribonucleotides (DNA), ribonucleotides (RNA), and peptide nucleic acids (PNAs)), used in the molecular structures of the present disclosure refers to a polymeric form of nucleotides of any length. Nucleotides comprise purine and pyrimidine bases, or other natural, chemically or biochemically modified, non-natural, or derivatized nucleotide bases. The backbone of the nucleic acid molecule (also referred to as a polynucleotide (comprising nucleotides)), can comprise sugars and phosphate groups, as may typically be found in DNA or RNA, or modified or substituted sugar or phosphate groups. A polynucleotide may comprise modified nucleotides, such as methylated nucleotides and nucleotide analogs.

Typically, a nucleic acid molecule will comprise phosphodiester bonds. However, nucleic acid molecules may comprise a modified backbone comprising, for example, phosphoramide, phosphorothioate, phosphorodithioate, O-methylphophoroamidite linkages, and peptide nucleic acid backbones and linkages. Other analog nucleic acids include those with positive backbones, non-ionic backbones, and non-ribose backbones. Nucleic acids containing one or more carbocyclic sugars are also included within the definition of nucleic acids. As will be appreciated by a person of skill in the art, all of these nucleic acid analogs may find use as helper strands or as part of a polynucleotide used to generate the nanostructures described herein. In addition, mixtures of naturally occurring nucleic acids and analogs can be made and are also suitable in the nanostructures described herein. PNAs include peptide nucleic acid analogs, which may have increased stability.

Thus, nucleic acid of various forms and conformations may be used for generating the three-dimensional nucleotide molecular structures described herein, including right-handed DNA, right-handed RNA, PNA, locked nucleic acid (LNA), threose nucleic acid (TNA), glycol nucleic acid (GNA), bridged nucleic acid (BNA), phosphorodiamidate morpholino oligo (PMO), as well as nucleotide analogues, such as non-Watson-Crick nucleotides dX, dK, ddX, ddK, dP, dZ, ddP, and ddZ.

In some embodiments, a three-dimensional molecular structure of the present disclosure comprises one or more distinct polymeric nucleic acid structures (e.g., at least 20, at least 50, at least 100, or at least 1000 or more distinct nucleic acid molecules). The nucleic acids may be single stranded or double stranded, or contain portions of both double stranded or single stranded sequence. The nucleic acid may be DNA, either or both genomic and cDNA, RNA or a hybrid, where the nucleic acid contains any combination of deoxyribo- and ribo-nucleotides, and any combination of bases, including uracil, adenine, thymine, cytosine, guanine, inosine, xanthine, hypoxanthine, isocytosine, isoguanine, and the like. Such nucleic acids comprise nucleotides and nucleoside and nucleotide analogs, and modified nucleosides such as amino modified nucleosides.

In some embodiments of the present disclosure, the nucleic acid nanostructure is DNA origami. DNA origami is a method of generating DNA artificially folded at nanoscale, creating an arbitrary three-dimensional shape that may be used as a scaffold for trapping inside, or capturing, an entity. Methods of producing DNA nanostructures of the origami type have been described, for example, in U.S. Pat. No. 7,842,793, which is hereby incorporated by reference in its entirety. DNA origami involves the folding of a long single strand of viral DNA (for example) aided by multiple smaller “staple” strands. These shorter strands bind the longer strand in various places, resulting in the formation of a 3D structure. The three-dimensional nucleotide molecular structures of the present disclosure may use numerous shorter single strands of nucleic acids (helper strands) (e.g., DNA) to direct the folding of a longer, single strand of polynucleotide (which is called, in DNA nanostructure nomenclature, the scaffold strand) into desired shapes, such as a nanoscale triangular subunit, that are usually between 100-5000 nm in diameter. A plurality of nanoscale triangular subunits have a configuration that allows those nanoscale triangular subunits to self-assemble in the form of a macromolecular icosahedral or cylindrical shell. The icosahedral or cylindrical shell may be on the order of about 100 nm to 5000 nm, but larger scaffolds of 10, 15, or 20 μm may also be achieved and used, depending on the context.

Nucleic acid nanotechnology makes use of the fact that, due to the specificity of Watson-Crick base pairing, only portions of the strands which are complementary to each other will bind to each other to form duplex. Construction of nucleic acid nanostructures has been described in several publications, including PCT Publication No. WO 2008/039254; U.S. Patent Application Publication No. 2010/0216978; PCT Publication No. WO 2010/0148085; U.S. Pat. Nos. 5,468,851; 7,842,793; Dietz et al., “Folding DNA Into Twisted and Curved Nanoscale Shapes,” Science 325:725-730 (2009); and Douglas et al., “Self-Assembly of DNA Into Nanoscale Three-Dimensional Shapes,” Nature 459:414 (2009); which are hereby incorporated by reference in their entirety, amongst others.

Natural or artificial sequences of DNA can be programmed to generate a three-dimensional (3D) structure. Usually, DNA-based nanostructures make use of a single strand of DNA which is induced into a 3D conformation by the binding of complementary, shorter DNA strands. In contrast, RNA folds into 3D by forming tertiary RNA motifs, based on RNA-RNA interactions within the same molecule. Nanostructures based on folded single-stranded DNA are also feasible. RNA duplexes are an alternative for generating RNA 3D structures.

In some embodiments, the three-dimensional nucleotide molecular structure of the present disclosure is a structure of joined tiles of DNA origami, in the form of the nanoscale triangular subunits, which self-assemble to form the icosahedral or cylindrical structure. Inducible nucleic acid nanostructures have been described, for example, by Andersen et al., “Self-Assembly of a Nanoscale DNA Box with a Controllable Lid,” Nature 459:73-77 (2009); Dietz et al., “Folding DNA Into Twisted and Curved Nanoscale Shapes,” Science 325:725-730 (2009); Voigt et al., “Single-Molecule Chemical Reactions on DNA Origami,” Nat. Nanotechnology 5:200-203 (2010); and Han et al., “DNA Origami with Complex Curvatures in Three-Dimensional Space,” Science 332:342-346 (2011); which are hereby incorporated by reference in their entirety). A software package for designing nucleic acid nanostructures is available at www.cdna.dk/origami.

As discussed in more detail below, three-dimensional nucleotide molecular structures described herein self-assemble to form a macromolecular shell. In some embodiments, the three-dimensional nucleotide molecular structure is a nanoscale triangular subunit. In some embodiments, all triangle bevel angles for a particular target shell are the same, however this need not always be the case.

In designing the three-dimensional nucleotide molecular structures and macromolecular shells described herein, iterative design may be used with, e.g., caDNAno (see Douglas et al., “Rapid Prototyping of 3D DNA-Origami Shapes with caDNAno,” Nucleic Acids Research 37:5001-5006 (2009), which is hereby incorporated by reference in its entirety) paired with elastic-network-guided molecular dynamics simulations (Maffeo et al., “De Novo Reconstruction of DNA Origami Structures Through Atomistic Molecular Dynamics Simulation,” Nucleic Acids Research 44:3013-3019 (2016), which is hereby incorporated by reference in its entirety) to produce candidate designs.

Exemplary DNA molecules for assembly of triangular subunits and macromolecular shells are disclosed in PCT Application Publ. No. WO 2022/261312 to Fraden et al., which is hereby incorporated by reference in its entirety. These triangular subunits can be used in the present application along with appropriate staple strands (creating an anchor) for binding one end of the one or more ss-DNA linkers to the triangular subunit. The ss-DNA linkers can be spaced apart along the length of each side of the triangular subunits to allow for flexibly linking together a plurality of triangular subunits. The optimal number of ss-DNA linker strands is 4 to 7 per side of a subunit, preferably 5 or 6 per side of a subunit.

The ss-DNA linkers each include a hybridizing segment at the other end of the ss-DNA linker and a spacer segment between the anchor and the hybridizing segment. The spacer segment length controls the spacing, flexibility, and degree of bending between a pair of triangular subunits that are joined together due to hybridization between mating hybridizing segments. As demonstrated herein, the length of the hybridizing segments can be optimized to promote assembly and the length of the spacer segments can be optimized to facilitate flexibility to accommodate irregularly shaped viruses.

The ss-DNA linkers between adjoining triangular subunits enable the macromolecular shell to bend and stretch around a virus or other particle intended to be encapsulated. In this sense, the nanoscale triangular subunits form a flexible shell due to the triangular subunits attaching to one another in a manner that conforms to the shape of the object to be encapsulated by the shell (virus or otherwise). ss-DNA is used to dictate the angles between adjacent subunits to create flexibility and curvature where needed. The degree of bending of regions of the shell formed by the triangular subunits can be controlled by the location and length of the spacer region of the one or more ss-DNA linkers that is intended to remain unpaired while linking the triangular subunits. In some embodiments, the flexibility of the shell can ultimately be controlled by the number of, e.g., polyT ss-DNA spacers introduced in the linker(s). Polythymidine (poly(T)) ss-DNA is a flexible polymer because it does not form any secondary structure.

In some embodiments, the spacer region contains between 2 to 10 unpaired bases, e.g., poly(T) containing 2, 3, 4, 5, 6, 7, 8, 9, or 10 thymidine bases.

In some embodiments, the spacer region contains between 11 to 20 unpaired bases, e.g., poly(T) containing 11, 12, 13, 14, 15, 16, 17, 18, 19, or 20 thymidine bases.

In some embodiments, the spacer region contains between 21 to 30 unpaired bases, e.g., poly(T) containing 21, 22, 23, 24, 25, 26, 27, 28, 29, or 30 thymidine bases.

In some embodiments, the spacer region contains more than 30 unpaired bases, e.g., poly(T) containing more than 30 thymidine bases.

In some embodiments, each ss-DNA linker includes a hybridizing segment, which is intended to pair with a mating hybridizing segment of a ss-DNA linker from an adjoining subunit. Hybridizing segments of DNA can be of varying lengths, including, for example, segments of 2 to 100 nucleotides, such as 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 bases (nucleotides) in length. Hybridizing segments of DNA can be between 2-10 nucleotides in length, or 2-20 nucleotides in length, or 10-20 nucleotides in length, or 20-30 nucleotides in length, or any range between 2 to about 100 nucleotides in length such as from 6 to 20 or 8 to 18 nucleotides in length.

Approximate target bevel angles for helical connectivity of triangle edges may be tuned in the vertices, and candidate designs may be encoded in DNA sequences using known methods of DNA origami (see Douglas et al., “Self-Assembly of DNA into Nanoscale Three-Dimensional Shapes,” Nature 459:414-418 (2009); Rothemund, “Folding DNA to Create Nanoscale Shapes and Patterns,” Nature 440:297-302 (2006); which are hereby incorporated by reference in their entirety) and self-assembled in one-pot reaction mixtures (see Wagenbauer et al., “How we Make DNA Origami,” Chembiochem: A European Journal of Chemical Biology (2017), which is hereby incorporated by reference in its entirety).

In some embodiments of the three-dimensional nucleotide molecular structure, the plurality of nanoscale triangular subunits self-assembles by lateral edge-to-edge stacking via base-pair stacking.

In some embodiments, each of the three edges of the nanoscale triangular subunits mates with only one of the three edges of an adjoining nanoscale triangular subunits.

In some embodiments, each of the three edges of the nanoscale triangular subunits mate with any of the three edges of an adjoining nanoscale triangular subunits. This affords simplicity and maximizes adaptability of the structural assemblies.

In some embodiments, the additional ss-DNA molecule is positioned along a base surface of the nanoscale triangular subunit.

In some embodiments, the three-dimensional nucleotide molecular structure is directed to coat a virus shell by targeting an inner surface (or base surface) of the nanostructure to the external surface of the virus particle. That can be achieved by tethering or linking a targeting moiety to the nanoscale triangular subunit along a base surface.

The targeting moiety can be a virus-specific receptor, antibody, active antibody fragment, nucleic acid aptamer, or peptide antibody mimic. These exemplary targeting moieties can be tethered to the base surface using a ss-DNA molecule covalently linked to the targeting moiety such that the targeting moiety has its active surface exposed on the base surface of the nanoscale triangular subunit.

As referred to herein, an “aptamer” is a relatively short nucleic acid (DNA, RNA, or a combination of both) sequence that binds with high avidity to a variety of proteins. Aptamers are generally about 25-40 nucleotides in length and have molecular weights in the range of about 18-25 kDa. Aptamers with high specificity and affinity for targets can be obtained by an in vitro evolutionary process termed SELEX (systemic evolution of ligands by exponential enrichment) (see, e.g., Zhang et al., Arch. Immunol. Ther. Exp. 52:307-315 (2004), which is hereby incorporated by reference in its entirety).

As referred to herein, “antibodies” relate to naturally derived, or naturally produced antibodies, which may be polyclonal or monoclonal. Alternatively, the antibodies may be synthetically produced by e.g., chemical synthesis, or recombinantly produced through the isolation of the specific mRNA from the respective antibody-producing cell or cell line. The specific mRNA shall then undergo standard molecular biology manipulations (obtaining cDNA, introducing the cDNA into expression vectors, etc.) in order to generate a recombinantly produced antibody.

The generation of polyclonal antibodies against proteins is a technique well known in the art, as described, e.g., a in Chapter 2 of Current Protocols in Immunology, John E. Coligan et al. (eds.), Wiley and Sons Inc., which is hereby incorporated by reference in its entirety.

The technique of generating monoclonal antibodies is described in many articles and textbooks, such as the above-noted Chapter 2 of Current Protocol in Immunology, Kohler and Milstein (Kohler and Milstein (1975) Nature 256:495-497), and in U.S. Pat. No. 4,376,110, which are hereby incorporated by reference in their entirety.

“Antibody” also includes both intact molecules as well as fragments thereof, such as, for example, scFv, Fv, Fab′, Fab, diabody, linear antibody, F(ab′)2 antigen binding fragment of an antibody which are capable of binding antigen (Wahl et al., “Improved Radioimaging and Tumor Localization with Monoclonal F(ab′)2,” J. Nucl. Med. 24:316-325 (1983), which is hereby incorporated by reference in its entirety.

In some embodiments, the three-dimensional DNA molecular structure comprises a targeting moiety that binds to a viral capsid protein. A “capsid protein” is a protein monomer. Capsid proteins can assemble together to form a capsomere (e.g., a pentamer of capsid proteins). A “capsomere” is a subunit of a viral capsid, which is an outer covering of protein that protects the genetic material of a virus such as, for example, human papillomavirus (HPV).

Capsids are broadly classified according to their structure. The majority of the viruses have capsids with either helical or icosahedral structure. The icosahedral shape, which has 20 equilateral triangular faces, approximates a sphere, while the helical shape resembles the shape of a spring, taking the space of a cylinder but not being a cylinder itself. The capsid faces may include one or more proteins.

Some viruses are enveloped, meaning that the capsid is coated with a lipid membrane known as the viral envelope. The envelope is acquired by the capsid from an intracellular membrane in the virus' host.

Once a virus has infected a cell and begins replicating itself, new capsid subunits are synthesized using the protein biosynthesis mechanism of the cell. In some viruses, including those with helical capsids and especially those with RNA genomes, the capsid proteins co-assemble with their genomes. In other viruses, especially more complex viruses with double-stranded DNA genomes, the capsid proteins assemble into empty precursor procapsids that include a specialized portal structure at one vertex. Through this portal, viral DNA is translocated into the capsid.

An external capsid protein is a capsid protein that is exposed on the surface of a VLP. A virus-like particle, or VLP, refers to an organized capsid-like structure (e.g., roughly spherical or cylindrical in shape) that comprises self-assembling ordered arrays capsomeres and does not include a viral genome. In some embodiments, the virus-like particles are morphologically and antigenically similar to authentic virions, but they lack viral genetic material (e.g., viral nucleic acid), rendering the particles non-infectious.

In some embodiments, the targeting moiety is tethered to a ss-DNA molecule that hybridizes to a discrete location along the base surface.

Design and location of targeting moieties to three-dimensional nucleotide structures described herein are described in more detail in PCT Application Publ. No. WO 2022/261312 to Fraden et al., which is hereby incorporated by reference in its entirety.

Another aspect of the present disclosure relates to flexible macromolecular shell formed by self-assembly of a plurality of the three-dimensional DNA molecular structures described herein.

All the various embodiments described above for the three-dimensional nucleotide molecular structures may also be applied to this aspect of the present disclosure.

In some embodiments of the flexible macromolecular shell, the three-dimensional DNA molecular structures are self-assembled by lateral edge-to-edge stacking via base-pair stacking, and the shell further comprises a linking agent that binds to two edge-to-edge stacked nanoscale triangular subunits.

In some embodiments, cylindrical shapes are formed. In some embodiments, icosahedral or spherical shapes are formed.

In some embodiments of forming a spherical or icosahedral shape, pseudo-symmetric triangular subunits may be designed based on multi-layer DNA origami concepts previously described (see e.g., Douglas et al., “Self-Assembly of DNA into Nanoscale Three-Dimensional Shapes,” Nature 459:414-418 (2009); Castro et al., “A Primer to Scaffolded DNA Origami,” Nature Methods 8:221-229 (2011); which are hereby incorporated by reference in their entirety).

In some embodiments, each side of a triangular subunit is the equivalent of one protein subunit of a natural viral capsid. Overall scale and type may be controlled by geometric instructions provided by the triangular subunits. These instructions are given by the choice of the length, the topological binding pattern (see Gerling et al., “Dynamic DNA Devices and Assemblies Formed by Shape-Complementary, Non-Base Pairing 3D Components,” Science 347:1446-1452 (2015), which is hereby incorporated by reference in its entirety), and the bevel angle of each triangular edge. In some embodiments, each triangular edge may represent, e.g., one protein, in which case the Caspar and Klug triangulation number gives the number of unique triangular edges required to build a particular icosahedral canvas shell. Hence, as described in International Patent Application No. PCT/US2022/032822, filed Jun. 9, 2022, which is hereby incorporated by reference in its entirety, T=1 and T=3 shells may both be built with a single triangle, with three identical edges for T=1 and three different edges for a T=3 shell. A T=4 shell requires two separate triangular subunits, for example, one triangle with three unique edges and another with three identical edges. A T=9 shell requires three different triangles, each having three unique edges. The greater the T number, the greater the overall number of triangles per target shell, given by 20T.

In some embodiments, macromolecular shells are coated (e.g., but without limitation, with a mixture of oligolysine and PEG oligolysine, as has been described, for example, in Ponnuswamy et al., “Oligolysine-Based Coating Protects DNA Nanostructures from Low-Salt Denaturation and Nuclease Degradation,” Nature Comm. 8:15654 (2017), which is hereby incorporated by reference in its entirety.

In some embodiments, the macromolecular shell is configured to encapsulate a filamentous virus particle, although encapsulation of other virus particles having different shapes (e.g., icosahedral) is also contemplated. A number of filamentous viruses are known to include many plant viruses as well as a number of animal viruses, including normally icosahedral animal viruses that nevertheless generate filamentous virus particles. Examples of normally filamentous virus particles include, without limitation, all Filoviridae such as Cuevavirus (e.g., Lloviu virus), Dianlovirus (e.g., Měnglà virus), Ebolavirus (e.g., Bombali virus, Bundibugyo virus, Reston virus, Sudan virus, Taï Forest virus, and Ebola virus), and Marburgvirus (e.g., Marburg virus, and Ravn virus). Others include Nipah and Hendra viruses. Examples of normally icosahedral viruses that generate filamentous forms include, without limitation, Influenza A and B viruses, Measles virus, Respiratory Syncytial virus, and African swine fever virus.

A further aspect of the present disclosure relates to a composition comprising a plurality of three-dimensional DNA molecular structures described herein in a carrier.

Another aspect of the present disclosure relates to a composition comprising a plurality of flexible macromolecular shells described herein in a carrier.

A further aspect of the present disclosure relates to a composition comprising a plurality of three-dimensional DNA molecular structures described herein and a plurality of flexible macromolecular shells as described herein in a carrier.

In some embodiments of the compositions of the present disclosure, the carrier is an aqueous carrier.

In some embodiments of the compositions of the present disclosure, the carrier is a pharmaceutically acceptable carrier.

In some embodiments, the pharmaceutically acceptable carrier is suitable for oral, mucosal, topical, or systemic delivery to a subject, such as a mammalian subject, including a human.

In some embodiments, the pharmaceutically acceptable carrier is suitable for delivery intranasally or by inhalation.

Three-dimensional DNA molecular structures described herein and/or macromolecular cylindrical shells described herein, and their compositions as described herein, can be used to encapsulate viral particles, including filamentous viral particles; inhibit viral infections; and treat individuals.

In other words, the present disclosure also relates to compositions containing the DNA origami nanostructures and the use of such compositions to cause filamentous viral particle encapsulation or to treat an individual for a viral infection by a filamentous virus. Such treatment can be prospective (i.e., to inhibit infection following exposure) or therapeutic (i.e., to treat an existing infection to minimize damage and shorten the infection and illness accompanying the same).

In the present disclosure, viruses can be trapped in, or coordinated by, pre-assembled shell segments (e.g., icosahedral shell segments) featuring sufficiently large apertures. Alternatively, protective shells can be formed directly on the surface of virus particles.

Such treatment is effected by administering nucleotide structures and shells described herein capable of encapsulating the filamentous virus particles to the subject. As used herein, the term “subject” refers to an animal, preferably a mammal such as a human.

Thus, another aspect of the present disclosure relates to a method of encapsulating a virus. This method involves providing a plurality of the three-dimensional DNA molecular structures described herein, and allowing said three-dimensional DNA molecular structures to self-assemble around a virus to form a shell, thereby encapsulating the virus.

A further aspect of the present disclosure relates to a method of inhibiting viral infection. This method involves encapsulating a virus with a flexible macromolecular shell as described herein, whereby the shell forms a physical barrier to inhibit viral infection of a cell otherwise susceptible to infection by the virus.

And yet another aspect of the present disclosure relates to a method of treating an individual for a viral infection. This method involves administering a composition described herein to an individual at a site of viral infection, where the flexible macromolecular shell forms a physical barrier that encapsulates viruses at the site of viral infection, thereby treating the individual.

In some embodiments, the three-dimensional nucleotide molecular structures and/or macromolecular shells described herein can be administered to the subject per se, or in a pharmaceutical composition where it is mixed with suitable carriers or excipients.

As used herein, a “pharmaceutical composition” refers to a preparation of one or more of the active ingredients described herein (i.e., a three-dimensional nucleotide molecular structure and/or macromolecular shell) with other chemical components such as physiologically suitable carriers and excipients. The purpose of a pharmaceutical composition is to facilitate administration of a compound to an organism.

As used herein, the term “active ingredient” refers to the individual three-dimensional nucleotide molecular structure and/or macromolecular shell formed by self-assembly of a plurality of the three-dimensional nucleotide molecular structures as described herein, as well as partial and complete assemblies thereof which are accountable for the intended biological effect.

In alternative embodiments, the individual three-dimensional nucleotide molecular structure and/or macromolecular shell formed by self-assembly of a plurality of the three-dimensional nucleotide molecular structures are used as a vehicle for delivering a pharmaceutical agent. Encapsulation and delivery of any known or later development pharmaceutical agent is contemplated.

The phrases “physiologically acceptable carrier” and “pharmaceutically acceptable carrier,” which may be used interchangeably, refer to a carrier or a diluent that does not cause significant irritation to an organism upon administration and does not abrogate the biological activity and properties of the administered active ingredient. An adjuvant is included under these phrases.

The term “excipient” used herein refers to an inert substance added to a pharmaceutical composition to further facilitate administration of an active ingredient. Examples of excipients include, without limitation, calcium carbonate, calcium phosphate, various sugars and types of starch, cellulose derivatives, gelatin, vegetable oils, and polyethylene glycols.

Techniques for formulation and administration of drugs may be found in the latest edition of “Remington's Pharmaceutical Sciences,” Mack Publishing Co., Easton, PA, which is hereby incorporated by reference in its entirety.

Suitable routes of administration include, for example, oral, rectal, transmucosal, especially transnasal, intestinal, or parenteral delivery, including intramuscular, subcutaneous, and intramedullary injections, as well as intrathecal, direct intraventricular, intravenous, intraperitoneal, intranasal, or intraocular injections. In some embodiments, routes of administration may include, without limitation, intranasal delivery and inhalation.

The pharmaceutical compositions described herein may be administered in a local rather than systemic manner, for example, via injection of the pharmaceutical composition directly into a tissue region of a patient.

Pharmaceutical compositions of the present disclosure may be manufactured by processes well known in the art, e.g., by means of conventional mixing, dissolving, granulating, dragee-making, levigating, emulsifying, encapsulating, entrapping, or lyophilizing processes.

Pharmaceutical compositions for use in the present disclosure may be formulated in a conventional manner using one or more physiologically acceptable carriers comprising excipients and auxiliaries, which facilitate processing of the active ingredients into preparations that can be used pharmaceutically. Proper formulation may be dependent on the route of administration chosen.

For injection, the active ingredients of the pharmaceutical composition may be formulated in aqueous solutions, such as in physiologically compatible buffers such as Hank's solution, Ringer's solution, or physiological salt buffer. For transmucosal administration, penetrants appropriate to the barrier to be permeated may be used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries as desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents, such as cross-linked polyvinyl pyrrolidone, agar, or alginic acid, or a salt thereof, such as sodium alginate, may be added.

Dragee cores are provided with suitable coatings. For this purpose, concentrated sugar solutions may be used that may optionally contain gum arabic, tale, polyvinyl pyrrolidone, carbopol gel, polyethylene glycol, titanium dioxide, lacquer solutions, and suitable organic solvents or solvent mixtures. Dyestuffs or pigments may be added to the tablets or dragee coatings for identification or to characterize different combinations of active compound doses.

Pharmaceutical compositions that can be used orally include push-fit capsules made of gelatin, as well as soft, sealed capsules made of gelatin and a plasticizer, such as glycerol or sorbitol. The push-fit capsules may contain the active ingredients in admixture with filler such as lactose, binders such as starches, lubricants such as tale or magnesium stearate, and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by inhalation, the active ingredients for use according to the present disclosure are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane, or carbon dioxide. In the case of a pressurized aerosol, the dosage may be determined by providing a valve to deliver a metered amount. Capsules and cartridges of, for example, gelatin for use in a dispenser may be formulated containing a powder mix of the compound and a suitable powder base, such as lactose or starch.

The pharmaceutical compositions described herein may be formulated for parenteral administration, e.g., by bolus injection or continuous infusion. Formulations for injection may be presented in unit dosage form, e.g., in ampules or in multi-dose containers with, optionally, an added preservative. The compositions may be suspensions, solutions, or emulsions in oily or aqueous vehicles, and may contain formulatory agents such as suspending, stabilizing, and/or dispersing agents.

Pharmaceutical compositions for parenteral administration include aqueous solutions of the active preparation in water-soluble form. Additionally, suspensions of the active ingredients may be prepared, as appropriate, with oily or water-based injection suspensions. Suitable lipophilic solvents or vehicles include fatty oils such as sesame oil, or synthetic fatty acid esters such as ethyl oleate, triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the active ingredients, to allow for the preparation of highly concentrated solutions.

In some embodiments, the active ingredient may be in powder form for constitution with a suitable vehicle, e.g., a sterile, pyrogen-free, water-based solution, before use.

Sustained-release (SR), extended-release (ER, XR, or XL), time-release or timed-release, controlled-release (CR), or continuous-release (CR or Contin) pills are tablets or capsules formulated to dissolve slowly and release a drug over time. Sustained-release tablets are formulated so that the active ingredient is embedded in a matrix of insoluble substance (e.g., acrylics, polysaccharides, etc.) such that the dissolving drug diffuses out through the holes in the matrix. In some SR formulations the matrix physically swells up to form a gel, so that the drug has first to dissolve in matrix, then exit through the outer surface.

The difference between controlled release and sustained release is that controlled release is perfectly zero order release. That is, the drug releases with time irrespective of concentration. On the other hand, sustained release implies slow release of the drug over a time period. It may or may not be controlled release.

Pharmaceutical compositions suitable for use in the context of the present disclosure include compositions where the active ingredients are contained in an amount effective to achieve the intended purpose. More specifically, a “therapeutically effective amount” means an amount of active ingredient(s) effective to prevent, alleviate, or ameliorate symptoms of a disorder or prolong the survival of the subject being treated.

Determination of a therapeutically effective amount is well within the capability of those skilled in the art, especially in light of the detailed disclosure provided herein.

For any preparation used in the methods of the present disclosure, the dosage or the therapeutically effective amount can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures, or experimental animals. The data obtained from in vitro, cell culture assays, and animal studies can be used in formulating a range of dosage for use in human. The dosage may vary depending upon the dosage form employed and the route of administration utilized. The exact formulation, route of administration, and dosage can be chosen by the individual physician in view of the patient's condition (see, e.g., Fingl et al., “The Pharmacological Basis of Therapeutics,” Ch. 1, p. 1 (1975), which is hereby incorporated by reference in its entirety).

Dosage amount and administration intervals may be adjusted individually to provide sufficient plasma or brain levels of the active ingredient to induce or suppress the biological effect (i.e., minimally effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks, or until cure is effected or diminution of the disease state is achieved.

The amount of a composition to be administered will, of course, be dependent on the subject being treated, the severity of the affliction, the manner of administration, the judgment of the prescribing physician, etc.

Compositions of the present disclosure may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser device may also be accompanied by a notice in a form prescribed by a governmental agency regulating the manufacture, use, or sale of pharmaceuticals, which notice is reflective of approval by the agency of the form of the compositions for human or veterinary administration. Such notice, for example, may include labeling approved by the U.S. Food and Drug Administration for prescription drugs or of an approved product insert. Compositions comprising a preparation of the present disclosure formulated in a pharmaceutically acceptable carrier may also be prepared, placed in an appropriate container, and labeled for treatment of an indicated condition, as further detailed above.

### EXAMPLES

The examples below are intended to exemplify the practice of embodiments of the disclosure but are by no means intended to limit the scope thereof.

### Example 1—Optimizing Conditions for Triangular Subunit Assembly

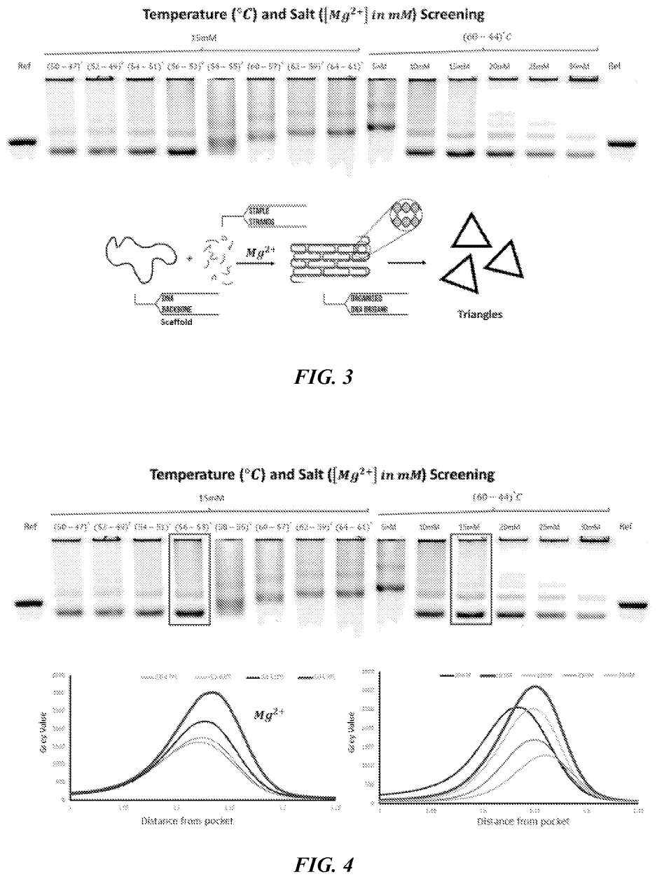

FIGS. 3 and 4 illustrate electrophoresis results assessing the effects of temperature and salt (Mg²⁺) concentration on triangular subunit assembly. DNA origami strands used are described in PCT Application Publ. No. WO 2022/261312 to Fraden et al., which is hereby incorporated by reference in its entirety. Although triangular subunits were formed over a range of temperature and salt (Mg²⁺) concentrations, specifically over temperatures from 44° C. to 60° C. and [Mg²⁺ ] from 10 to 30 mM, optimal results were achieved using 53° C. to 56° C. and [Mg²⁺ ] of about 15 mM.

### Example 2—Flexibly Linked Triangular Subunit Assembly on Cholesterol Particles

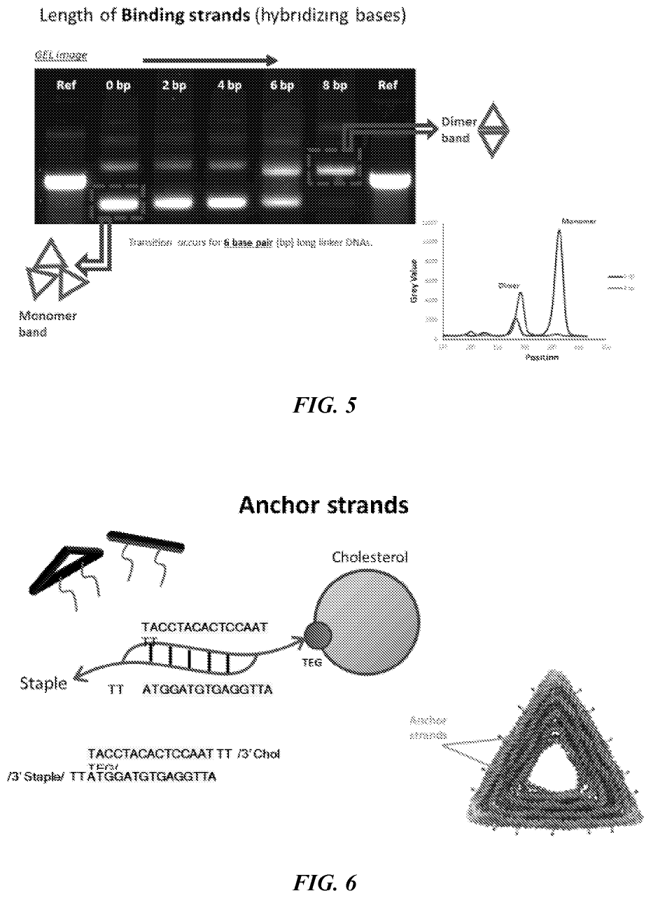

FIGS. 5-8 illustrate the joining together of triangular subunits to form dimers and larger subunit assemblies using ss-DNA linkers to form the assemblies on the surface of cholesterol particles. To facilitate this proof of principle, cholesterol particles bearing TACCTACACTCCAATTT-triethyleneglycol (“TEG”)-linked DNA strands (SEQ ID NO: 2) were used along with triangular subunits bearing ATTGGAGTGTAGGTATT ss-DNA linker strands (SEQ ID NO: 3) and ATTGGAGTGTAGGTATTTTTTTTTTTTTTTTTTTT ss-DNA linker strands (SEQ ID NO: 4), which were secured to the triangular subunits using staple strands.

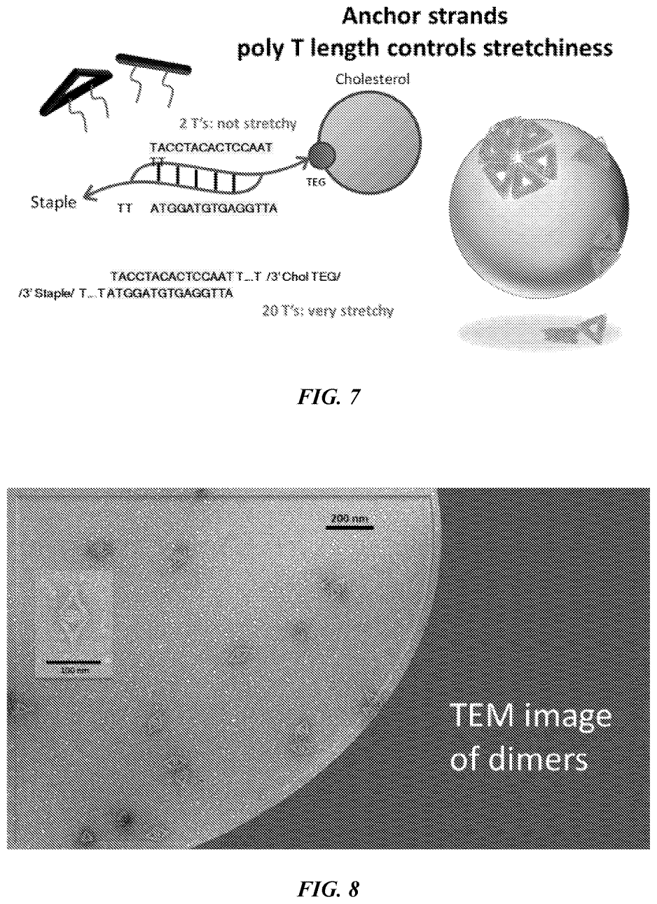

FIG. 5 shows that ss-DNA linkers having a hybridizing segment of 6 or 8 bp contributed to dimer formation, whereas hybridizing segments of 2 or 4 bp show only background levels of dimer formation. The longer 20T segment in SEQ ID NO: 4 is more flexible that the shorter 2T segment in SEQ ID NO: 3 (FIGS. 6-7).

FIG. 8 is a transmission electron microscopy (“TEM”) image illustrating dimer assembly. The ss-DNA linker strands have a hybridization segment containing 6 bp. Inset is an enlarged image showing a single dimer assembly.

### Example 3—Flexibly Linked Triangular Subunits Form Open Shell for Encapsulation of Virus Particles

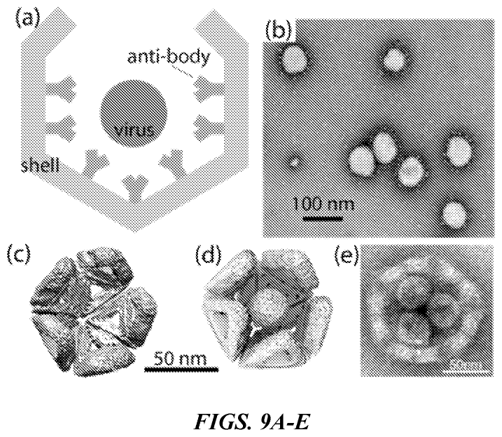

An application of the self-limiting assembly is to use DNA origami shells for an unconventional antiviral concept: rather than targeting viral proteins individually by antibodies as is currently the norm, their approach is to engulf whole viruses within de novo designed shells to block interactions between virus and host cells (FIG. 9A). The key advantage of this approach is that blocking infection requires just one binding event between the virus and encapsulating shell. In contrast, the traditional antibody approach for covid-19 requires that most of the ~100 viral surface Spike glycoproteins be bound due to their large functional redundancy in cell attachment and endosomal membrane fusion. For this experiment, a designed pseudotyped vesicular stomatitis virus (VSV) displaying covid-19 Spike protein in place of the VSV glycoprotein (VSV-CoV2S) (FIG. 9B) will be used.

Like that shown in FIGS. 9C-E, the cellular receptor for SARS-CoV-2 with a modification that will allow it to be conjugated to the DNA origami shell subunits will be used to modify the flexibly linked triangular subunits as described in the preceding Examples. To confirm efficacy, coated virus-sized polystyrene beads functionalized with the Covid-19 Spike protein will serve as a model system for the targeted encapsulation of viruses with DNA origami to facilitate precise tuning of DNA origami interaction strengths to optimize virus trapping.

Having thus described the basic concept of the invention, it will be rather apparent to those skilled in the art that the foregoing detailed disclosure is intended to be presented by way of example only, and is not limiting. Various alterations, improvements, and modifications will occur and are intended to those skilled in the art, though not expressly stated herein. These alterations, improvements, and modifications are intended to be suggested hereby, and are within the spirit and scope of the invention. Additionally, the recited order of processing elements or sequences, or the use of numbers, letters, or other designations therefore, is not intended to limit the claimed processes to any order except as may be specified in the claims. Accordingly, the invention is limited only by the following claims and equivalents thereto.

What is claimed:

1. A three-dimensional DNA molecular structure comprising:
one or more DNA strands folded in the form of a nanoscale triangular subunit having a configuration that allows a plurality of said nanoscale triangular subunits to self-assemble in the form of a flexible macromolecular shell, wherein the nanoscale triangular subunits are connected together by hybridization of one or more single-stranded DNA (“ss-DNA”) linkers on adjoining triangular subunits, which retain a number of unpaired bases whereby the unpaired bases of the hybridized ss-DNA linkers enables the macromolecular shell to bend and stretch around a virus or other particle intended for encapsulation.

2. The three-dimensional DNA molecular structure according to claim 1, wherein the plurality of said nanoscale triangular subunits self-assemble by lateral edge-to-edge stacking via base-pair stacking.

3. The three-dimensional DNA molecular structure according to claim 2, wherein each of the three edges of the nanoscale triangular subunits mates with only one of the three edges of an adjoining nanoscale triangular subunits.

4. The three-dimensional DNA molecular structure according to any one of the preceding claims, wherein each ss-DNA linker includes a hybridizing segment, which is intended to pair with a mating hybridizing segment of a ss-DNA linker from an adjoining subunit.

5. The three-dimensional DNA molecular structure according to claim 4, wherein the hybridizing segment comprises between 2 to 100 nucleotides.

6. The three-dimensional DNA molecular structure according to claim 5, wherein an additional ss-DNA molecule is positioned along a base surface of the nanoscale triangular subunit.

7. The three-dimensional DNA molecular structure according to any one of claims 1 to 6, further comprising a targeting moiety linked to the nanoscale triangular subunit along a base surface.

8. The three-dimensional DNA molecular structure according to claim 7, wherein the targeting moiety is an antibody, active antibody fragment, nucleic acid aptamer, or peptide antibody mimic.

9. The three-dimensional DNA molecular structure according to claim 7, wherein the targeting moiety binds to a viral capsid protein.

10. The three-dimensional DNA molecular structure according to claim 7, wherein the targeting moiety is tethered to a ss-DNA molecule that hybridizes to a discrete location along the base surface.

11. The three-dimensional DNA molecular structure according to any one of claims 1 to 10, wherein the nanoscale triangular subunit comprises more than one DNA strand.

12. A flexible macromolecular shell formed by self-assembly of a plurality of the three-dimensional DNA molecular structures according to any one of claims 1 to 11.

13. The flexible macromolecular shell according to claim 12, wherein the three-dimensional DNA molecular structures are self-assembled by lateral edge-to-edge stacking via base-pair stacking, and the shell further comprises a linking agent that binds to two edge-to-edge stacked nanoscale triangular subunits.

14. The flexible macromolecular shell according to claim 13, wherein the linking agent is an antibody, active antibody fragment, nucleic acid aptamer, or peptide antibody mimic.

15. The flexible macromolecular shell according to any one of claims 12 to 14, wherein the shell has a 5,0 lattice structure, a 5,3 lattice structure, or a 5,5 lattice structure.

16. The flexible macromolecular shell according to claim 16, wherein the shell is configured to encapsulate a virus.

17. A composition comprising a plurality of three-dimensional DNA molecular structures according to any one of claims 1 to 11 in a carrier.

18. A composition comprising a plurality of flexible macromolecular shells according to any one of claims 12 to 16 in a carrier.

19. A composition comprising a plurality of three-dimensional DNA molecular structures according to any one of claims 1 to 11 and a plurality of flexible macromolecular shells according to any one of claims 12 to 16 in a carrier.

20. The composition according to any one of claims 17 to 19, wherein the carrier is an aqueous carrier.

21. The composition according to any one of claims 17 to 19, wherein the carrier is a pharmaceutically acceptable carrier.

22. The composition according to claim 21, wherein the pharmaceutically acceptable carrier is suitable for oral, mucosal, topical, or systemic delivery.

23. The composition according to claim 21, wherein the pharmaceutically acceptable carrier is suitable for delivery intranasally or by inhalation.

24. A method of encapsulating a virus, said method comprising:
providing a plurality of the three-dimensional DNA molecular structures according to any one of claims 1 to 11, and allowing said three-dimensional DNA molecular structures to self-assemble around a virus to form a shell, thereby encapsulating the virus.

25. A method of inhibiting viral infection comprising:
encapsulating a virus with a flexible macromolecular shell according to any one of claims 12 to 16, whereby the shell forms a physical barrier to inhibit viral infection of a cell otherwise susceptible to infection by the virus.

26. The method according to claim 24 or 25, wherein said method is carried out in vitro.

27. The method according to claim 24 or 25, wherein said method is carried out in vivo.

28. The method according to claim 25 to 26, wherein the cell is an animal cell.

29. The method according to claim 28, wherein the animal cell is a mammalian cell or an avian cell.

30. The method according to claim 25 to 26, wherein the cell is a plant cell.

31. A method of treating an individual for a viral infection, said method comprising:
administering a composition according to one of claims 21 to 23 to an individual at a site of viral infection, wherein the flexible macromolecular shell forms a physical barrier that encapsulates viruses at the site of viral infection, thereby treating the individual.

32. The method according to claim 31, wherein said administering is by oral, mucosal, topical, or systemic delivery.

33. The method according to claim 31, said administering is carried out intranasally or by inhalation.
